(19) United States
(12) Patent Application Publication
(10) Pub. No.: US 2018/0369503 A1
(43) Pub. Date: Dec. 27, 2018

(54) METHOD AND SYSTEM FOR REDUCING PATIENT STRESS DURING PROLONGED SUBCUTANEOUS PHARMACEUTICAL INJECTION

(71) Applicant: YING ZHAO, LEXINGTON, MA (US)

(72) Inventor: YING ZHAO, LEXINGTON, MA (US)

(21) Appl. No.: 16/048,067

(22) Filed: Jul. 27, 2018

(51) Int. Cl. A61M 5/42; A61M 5/142; A61M 5/32

(52) U.S. Cl. CPC A61M 5/42; A61M 2005/206; A61M 5/3287; A61M 5/14244

(57) ABSTRACT

A method for reducing patient stress during prolonged subcutaneous pharmaceutical injection wherein a flexible, nonmetallic sleeve is provided for pharmaceutical delivery. A penetration needle, disposed in the sleeve, allows the flexible sleeve to penetrate a patient's skin. Once the flexible, nonmetallic sleeve has penetrated a patient's skin the penetration needle is removed and a delivery needle is introduced to an upper and of the sleeve.

### BACKGROUND

Application of pharmaceuticals has come a long way in the past couple of decades. One of the most liberating mechanisms for introducing a pharmaceutical into a human recipient is a concept based on a “wearable injector”. A wearable injector includes a reservoir, which contains a quantity of pharmaceutical agent. A wearable injector also includes a delivery timing mechanism which provides a means for introducing a particular quantity of pharmaceutical agent into a recipient over a particular interval of time.

It can be appreciated that a wearable injector allows a patient to receive sustained application of a pharmaceutical agent over a much longer period of time, especially when compared to a single injection by hypodermic needle. Despite of the advantages provided by a wearable injector, all wearable injectors available today continue to rely upon a rigid needle, which penetrates a patient's skin and stays in place during the entire duration of pharmaceutical agent delivery.

It can be appreciated that the rigid needle is likely to cause skin irritation proximate to the penetration point. Accordingly, severe patient discomfort may result. Patient discomfort is but one potential indication when using a rigid needle to maintain penetration of a patient's skin over an extended duration of time. Irritation may lead to inflammation. In yet other cases, the rigid needle may tear at the skin causing repeated exposure to microbial infection.

Another issue with the use of a wearable injector is the fact that the injector itself may occasionally need to be repositioned. For example, a wearable injector, once its rigid needle has penetrated a patient's skin, is typically affixed to the skin by some form of adhesive. When such adhesive binding to the patient's skin fails, the wearable injector must be removed from the skin and the rigid needle is then exposed to sources of infection. And even though the rigid needle is exposed to sources of infection, it is typically reused to re-penetrate the skin in order to re-affix the wearable injector to the patient.

### BRIEF DESCRIPTION OF THE DRAWINGS

Several alternative embodiments will hereinafter be described in conjunction with the appended drawings and figures, wherein like numerals denote like elements, and in which:

FIG. 1A is a flow diagram that depicts one example method of reducing the stress associated with prolonged pharmaceutical application:

FIGS. 1B through 1D are pictorial diagrams that depict one example application of the present method; FIG. 2 is a flow diagram that depicts one alternative example method for fixing an upper end of the sleeve in a holder;

FIG. 3 is a flow diagram that depicts one alternative example method for providing a penetration needle into the sleeve;

FIG. 4 is a flow diagram that depicts one alternative example method for accepting a delivery needle into the sleeve;

FIG. 5 is a flow diagram that depicts one alternative example method for accepting a delivery needle into the sleeve substantially contemporaneous with receiving a delivery reservoir into a holder;

FIG. 6 is a flow diagram that depicts one alternative example method for affixing the holder to a patient's skin;

FIG. 7 is a flow diagram that depicts one alternative example method wherein a seal is maintained between a delivery needle and the upper end of the sleeve;

FIG. 8 is a flow diagram that depicts one alternative example method wherein an upper end of the sleeve is sealed when the penetration needle is removed;

FIG. 9 is a flow diagram that depicts yet another example method wherein a delivery reservoir is restrained in a delivery holder:

FIG. 10 is a flow diagram that depicts one alternative example method wherein a holder can be changed while maintaining penetration of a delivery needle in the sleeve and the sleeve in a patient;

FIG. 11 is a pictorial diagram that illustrates one example embodiment of a system for reducing patient stress during prolonged subcutaneous pharmaceutical injection;

FIG. 12 is a pictorial diagram illustrates assembly of one alternative example embodiment of a system for reducing patient stress during prolonged transdermal pharmaceutical injection;

FIGS. 13 and 14 are pictorial diagrams that illustrate insertion of a pharmaceutical injection device into the holder;

FIG. 15 is a pictorial diagram that depicts an underside of one example embodiment of a holder; and

FIG. 16 is a pictorial diagram that illustrates how a flexible sleeve is released from a holder when the holder needs to be replaced.

### DETAILED DESCRIPTION

In the interest of clarity, several example alternative methods are described in plain language. Such plain language descriptions of the various steps included in a particular method allow for easier comprehension and a more fluid description of a claimed method and its application. Accordingly, specific method steps are identified by the term “step” followed by a numeric reference to a flow diagram presented in the figures, e.g. (step 5). All such method “steps” are intended to be included in an open-ended enumeration of steps included in a particular claimed method. For example, the phrase “according to this example method, the item is processed using A” is to be given the meaning of “the present method includes step A, which is used to process the item”. All variations of such natural language descriptions of method steps are to be afforded this same open-ended enumeration of a step included in a particular claimed method.

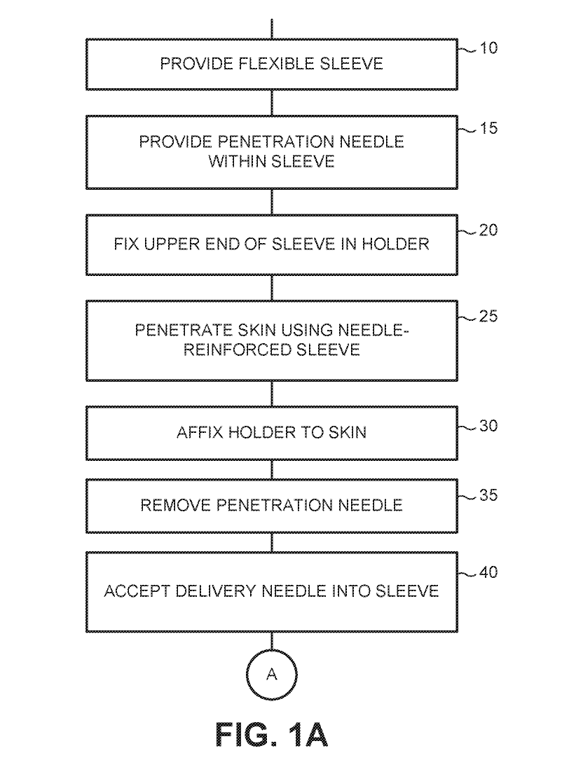

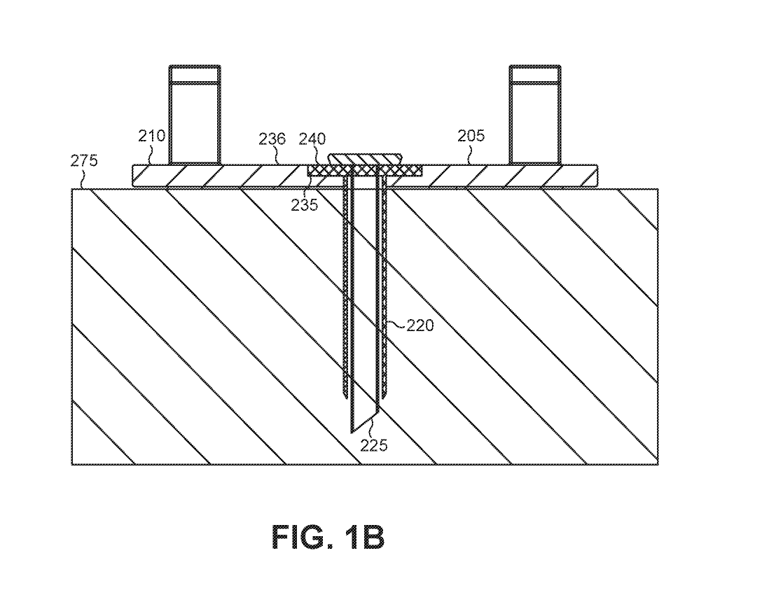

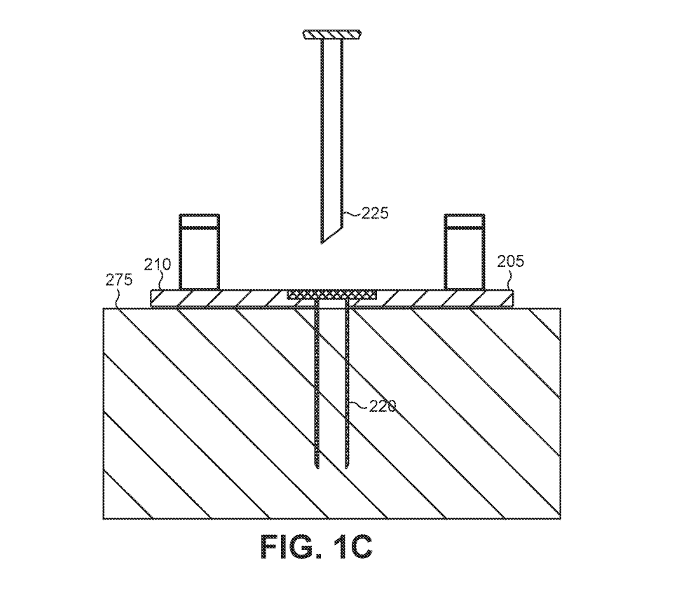

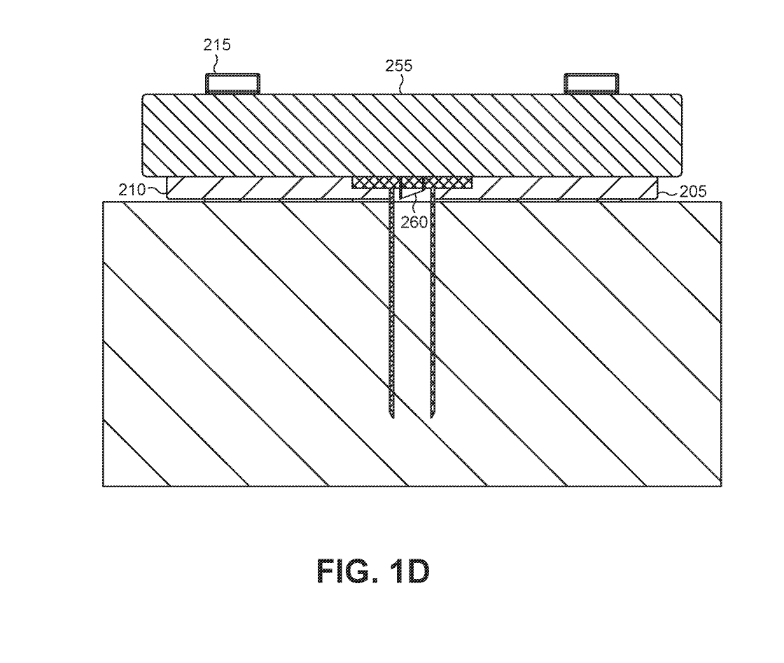

FIG. 1A is a flow diagram that depicts one example method of reducing the stress associated with prolonged pharmaceutical application. According to this example method, stress upon a patient can be reduced during subcutaneous pharmaceutical injection by method that comprises providing a flexible, nonmetallic sleeve (step 10), providing a penetration needle within the sleeve (step 15), fixing an upper end of the sleeve in a holder (step 20), penetrating the skin using the sleeve reinforced with the penetration needle (step 25), affixing the holder to a patient's skin (step 30), removing the penetration needle from the sleeve (step 35) and accepting a delivery needle into the sleeve (step 40).

FIGS. 1B through 1D are pictorial diagrams that depict one example application of the present method. It should be appreciated that, according to one illustrative use case, the flexible sleeve 220 penetrates a patient's skin 275 in that it is concentric with the penetration needle 225 provided therein. The penetration needle 225 protrudes beyond the flexible, nonmetallic sleeve 220. Once the penetration needle 225 penetrates the patient's skin, the flexible sleeve 220 follows along.

FIG. 1C shows that once the flexible sleeve 220 penetrates the skin 275, the penetration needle 225 is withdrawn from the sleeve 220. FIG. 1D illustrates that a, once a wearable injector 255 is snapped into a holder, which in this illustrative embodiment includes a first-half 205 and a second-half 210, a delivery needle 260 included in the wearable injector 255 is received into the flexible sleeve 220.

FIG. 2 is a flow diagram that depicts one alternative example method for fixing an upper end of the sleeve in a holder. It should be appreciated that, according to this example method, fixing an upper end of the sleeve and a holder comprises supporting a lower surface of a collar included in the sleeve with a recessed upper surface included in a holder (step 45). This alternative method also includes a step for holding the top of the collar substantially flush with an upper surface of the holder (step 50). In this manner, the collar, which according to one example embodiment is integral with the upper end of the sleeve, is prevented from slipping downward through the holder.

FIG. 1B shows one example structure and wherein the flexible sleeve 220 includes a collar 240. As seen in this figure, a recessed feature includes a surface 235 that is set downward from a top surface 236 included in the holder. It should likewise be appreciated that the holder, according to one alternative embodiment, includes a first-half 205 and a second-half 210.

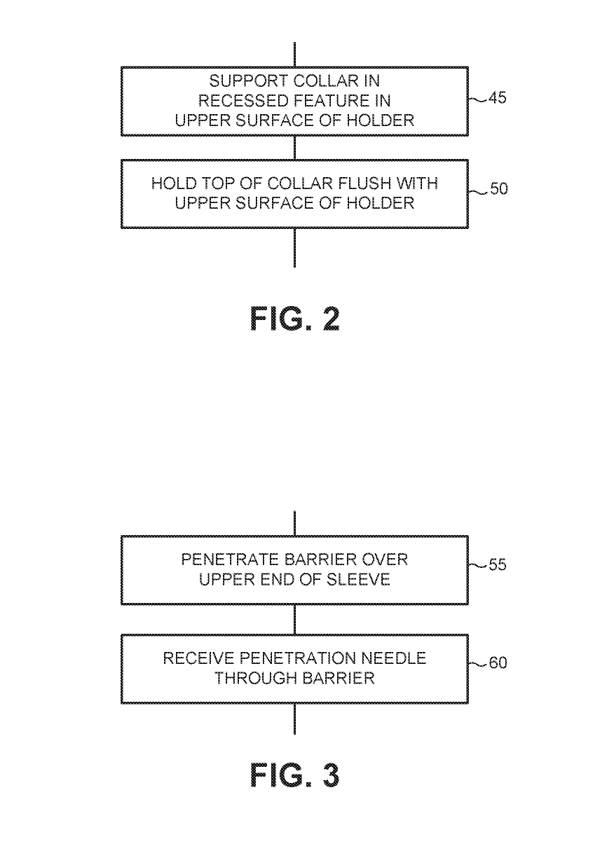

FIG. 3 is a flow diagram that depicts one alternative example method for providing a penetration needle into the sleeve. According to this alternative example method, providing a penetration needle into the sleeve comprises penetrating a barrier over the upper end of the sleeve (step 55) and receiving the penetration needle through the barrier (step 60).

FIG. 1B shows that, according to one example embodiment, the flexible sleeve 220 includes a collar integrated there with at the flexible sleeve's 220 upper end. It should be appreciated that, according to yet another alternative example embodiment, the collar 240 includes a barrier at the upper end of the collar 240. According to one illustrative use case, this example alternative method provides for penetrating such a barrier when the penetration needle 225 is inserted into the sleeve 220. It should likewise be appreciated that the barrier, according to yet another alternative example embodiment, provides for substantially preventing ingress of unwanted matter from an outside environment into the flexible nonmetallic sleeve 220.

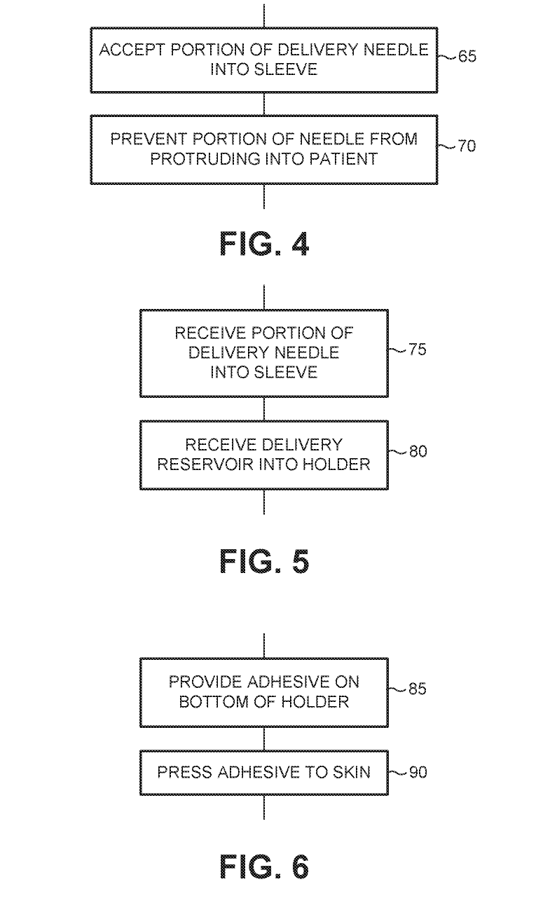

FIG. 4 is a flow diagram that depicts one alternative example method for accepting a delivery needle into the sleeve. According to this alternative example method, accepting a delivery needle into the sleeve comprises accepting a portion of the delivery needle into the sleeve (step 65) and preventing the received portion of the delivery needle from protruding into a patient (step 70).

FIG. 1B also shows that, according to yet another alternative example embodiment, the thickness of the holder is sized in order to prevent the delivery needle from protruding into the sleeve 220 to such extent as to extend into a patient's skin.

FIG. 5 is a flow diagram that depicts one alternative example method for accepting a delivery needle into the sleeve substantially contemporaneous with receiving a delivery reservoir into a holder. As depicted in the figure, one alternative example method for accepting a delivery needle comprises receiving a portion of the delivery needle into the sleeve (step 75) and receiving a delivery reservoir into a holder (step 80). It should be appreciated that, according to this alternative example method, the delivery reservoir is received into the holder substantially contemporaneous with receiving a portion of the delivery needle into the sleeve.

It should be appreciated that, according to one illustrative use case, as a delivery reservoir, which is typically included in a wearable injector, is positioned into a holder, the delivery needle 260 begins to penetrate a barrier included over the top end of the collar 240 that is integral to the flexible sleeve 220. As the wearable injector is further positioned into the holder, which in this alternative example embodiment includes a first-half 205 and a second-half 210, the delivery needle 260 is further situated into the flexible, nonmetallic sleeve 220.

FIG. 6 is a flow diagram that depicts one alternative example method for affixing the holder to a patient's skin. According to this alternative example method, affixing the holder to a patient's skin comprises providing an adhesive on a bottom surface of the holder (step 85) and then pressing the adhesive to the skin (step 90). It should be appreciated that, according to one illustrative use case, the holder must be held on a patient's skin for a duration of time. Accordingly, this alternative example method provides for a means for maintaining the holder in position on a patient. As a result, a wearable injector positioned and retained in the holder can be worn by a patient for such duration as required to administer a pharmaceutical over a particular interval of time.

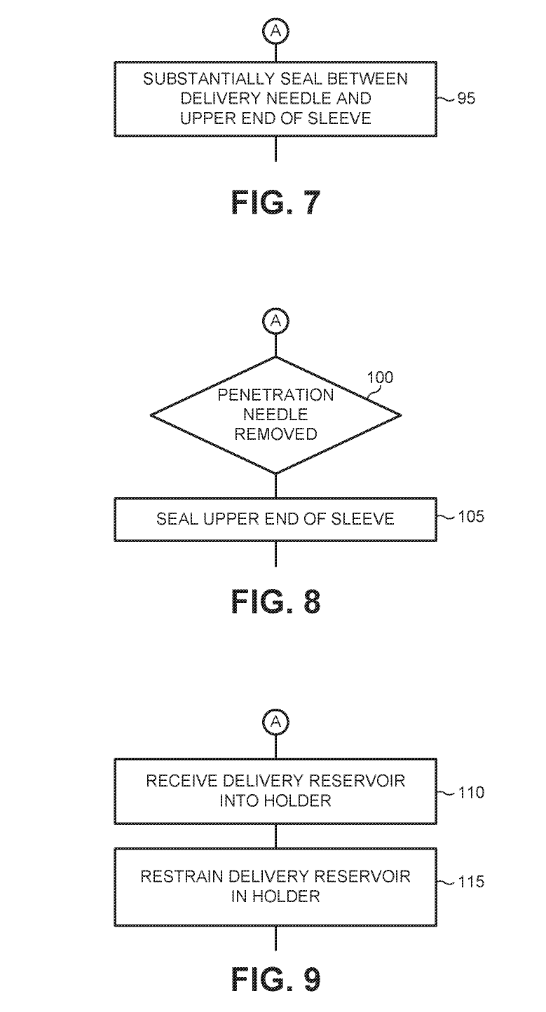

FIG. 7 is a flow diagram that depicts one alternative example method wherein a seal is maintained between a delivery needle and the upper end of the sleeve. It should be appreciated that, according to this example method, an additional step is included for substantially sealing between the delivery needle and the upper end of the sleeve (step 95). It should be appreciated that once a delivery needle is disposed in the upper end of the sleeve, it is important to maintain a sterile environment so that infection cannot be introduced into a patient. It should be appreciated that, according to one illustrative use case, maintaining a seal between the delivery needle and the upper end of the sleeve is accomplished by a barrier that is integral to the top end of the sleeve and which exhibits elasticity sufficient to conform in a substantially hermetic way around the perimeter of the delivery needle.

FIG. 8 is a flow diagram that depicts one alternative example method wherein an upper end of the sleeve is sealed when the penetration needle is removed. One alternative example method further comprises a step for sealing the upper end of the sleeve (step 105) when the penetration needle is removed (step 100). It should be appreciated that, when the penetration needle is removed from the sleeve, is important to maintain a sterile environment within the sleeve so that infection cannot be introduced into a patient. As such, one illustrative application of the present method is accomplished where a barrier is made integral to the top of a sleeve and wherein said barrier exhibit sufficient elasticity to conform to the penetration needle and also exhibit sufficient elasticity to substantially seal itself when the penetration needle is removed. In this sense, the barrier is “self-healing”.

FIG. 9 is a flow diagram that depicts yet another example method wherein a delivery reservoir is restrained in a delivery holder. It should be appreciated that, according to one alternative example method, a delivery reservoir (e.g. a wearable injector) is introduced into a holder (step 110). According to this alternative example method, the delivery reservoir is then restrained in the holder (step 115).

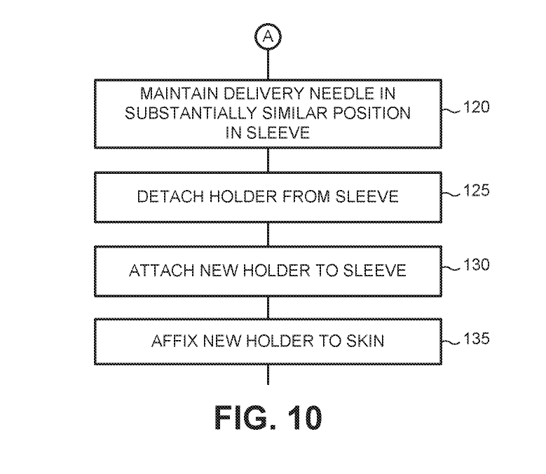

FIG. 10 is a flow diagram that depicts one alternative example method wherein a holder can be changed while maintaining penetration of a delivery needle in the sleeve and the sleeve in a patient. It should be appreciated that, according to this alternative example method, one included step provides for maintaining the delivery needle in a substantially similar position in the sleeve (step 120). It should be appreciated that, according to various illustrative use cases, a user may need to change the holder for various reasons. One such reason is that any adhesive that is included on an underside of the holder has ceased to be effective. Again, this is only one illustrative use case and it is not intended to limit the scope of the claims appended hereto.

According to this alternative example method, the holder is then detached from the sleeve in a further included step (step 125). In yet an additional step, a new holder is then attached to the sleeve (step 130). It should be noted that, according to various illustrative use cases, detaching the holder from the sleeve, as in step 125, and then attaching a new holder to the same sleeve is accomplished while the sleeve is maintained in its inserted position and the delivery needle is also maintained in position in the sleeve. Once the new holder is attached to the sleeve, it is affixed to the skin in an additional included step (step 135).

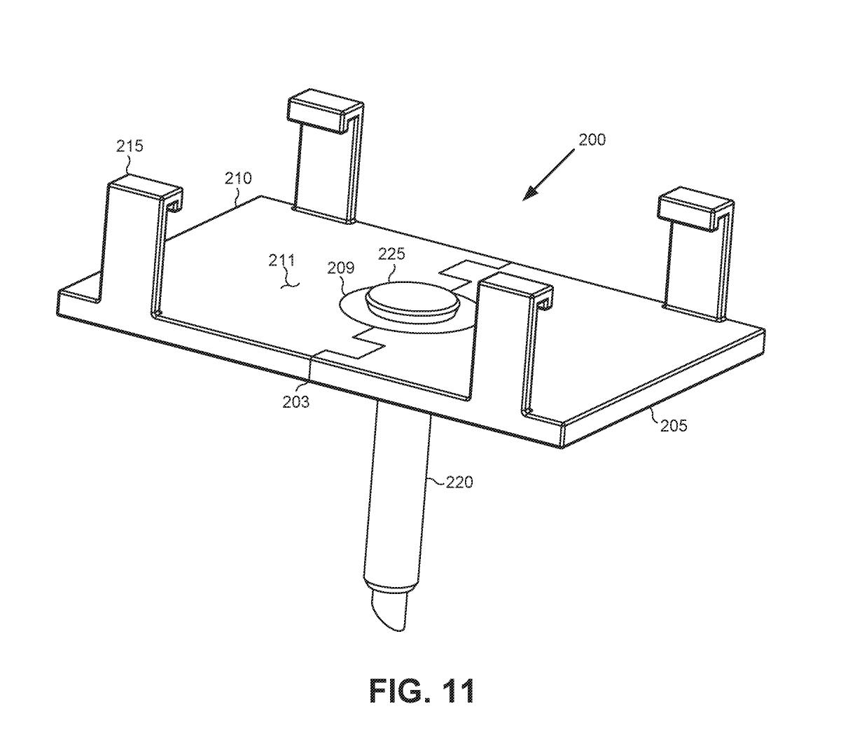

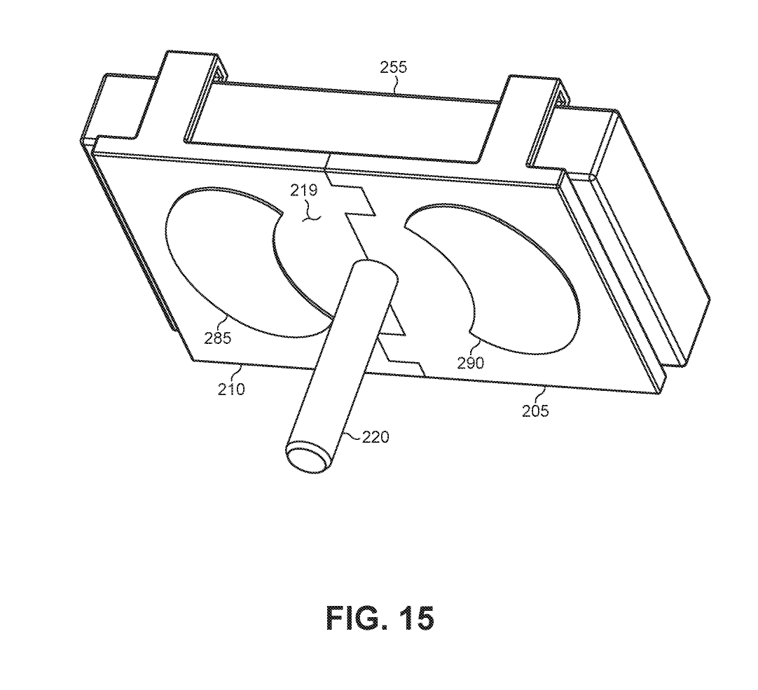

FIG. 11 is a pictorial diagram that illustrates one example embodiment of a system for reducing patient stress during prolonged subcutaneous pharmaceutical injection. According to this example embodiment, such a system 200 comprises a holder 203, and an adhesive surface (see 285, 290 in FIG. 15) disposed on a portion of the bottom surface of the holder. It should be appreciated that, according to this example embodiment, the holder includes a top surface 211 and a bottom surface (see 219 in FIG. 15) and also includes an opening 209 for holding a flexible sleeve.

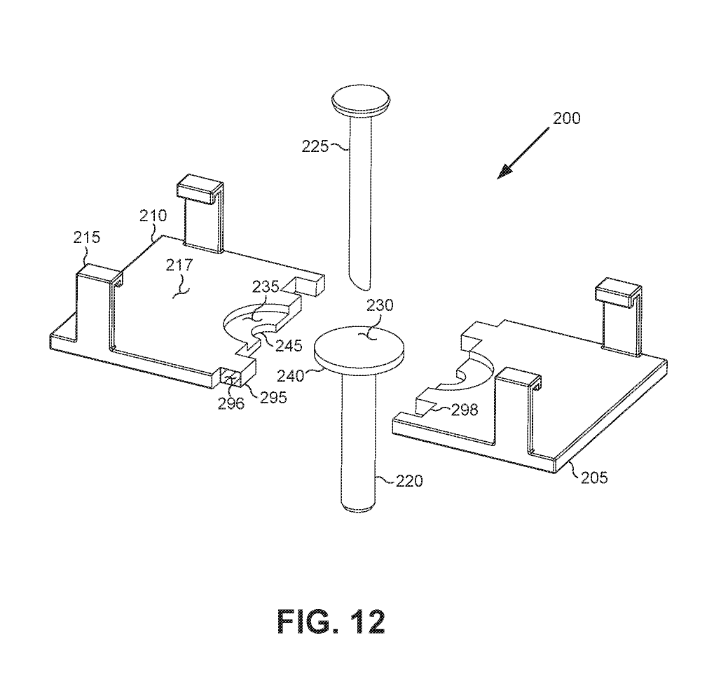

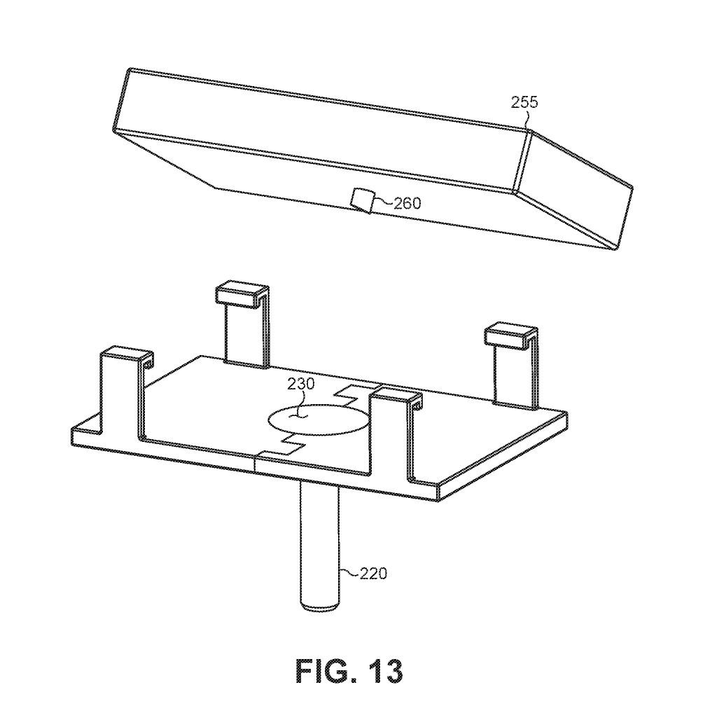

FIG. 12 is a pictorial diagram that illustrates assembly of one alternative example embodiment of a system for reducing patient stress during prolonged transdermal pharmaceutical injection. One alternative example embodiment of the system 200 further includes a flexible sleeve 220 that runs through the top and bottom surfaces of the holder 203. It should be appreciated that, according to this alternative example embodiment, the flexible sleeve 220 is disposed in the opening included in the holder 203 for said purpose. According to this alternative example embodiment, the flexible sleeve 220 includes an upper end which itself includes a collar 240. The collar 240 is sized in order to fit in a depression included in the top surface of the holder 203.

According to one alternative example embodiment, the flexible sleeve 220 further includes a seal-membrane 230 disposed across the upper end of the collar 240. It should be appreciated that the seal-membrane comprises a pliable material to allow penetration by a penetration needle 225. The pliable material, according this alternative embodiment, provides for a self-healing mechanism so that the seal-membrane 230 will substantially reseal when the penetration needle 225 is withdrawn from the flexible, nonmetallic sleeve 220. As such, the internal volume of the flexible sleeve 220 is substantially sealed against unwanted foreign matter.

According to one alternative example embodiment, the holder 203 comprises a first portion 210 and a second portion 205. In this alternative example embodiment, the first portion 210 and the second portion 205 include an interlock structure which is used to substantially maintain the position of the first portion 210 relative to the second portion 205 when said first and second portions are coupled together.

According to yet another alternative example embodiment, the interlock structure comprises a tongue and groove mechanism. In this alternative example embodiment, a first portion 210 of the holder 203 includes a tongue 295 and the second portion of the holder 205 includes a corresponding groove 298. It should likewise be appreciated that, in order to maintain the position of the first portion 210 relative to the second portion 205 when the two portions are coupled together, this alternative example embodiment includes a rough surface 296 included on a side of the tongue 295. It should likewise be appreciated that the width of the groove 298 is sized so as to make a friction fit with the rough surface 296 included in the tongue 295.

According to yet another alternative example embodiment, the opening 245 included in the holder 203 is shrouded by a depressed region 235. The depressed region 235 provides for a depth so as the collar 240 included at the top end of the sleeve 220 is supported at the depressed region 235. According to one alternative embodiment, the depth of the depressed region corresponds to the thickness of the collar 240 included at the top end of the flexible-nonmetallic sleeve 220. Thus, according to this alternative embodiment, the top surface of the collar, which comprises the seal-membrane 230, sits substantially flush with a top surface 217 of the holder 203.

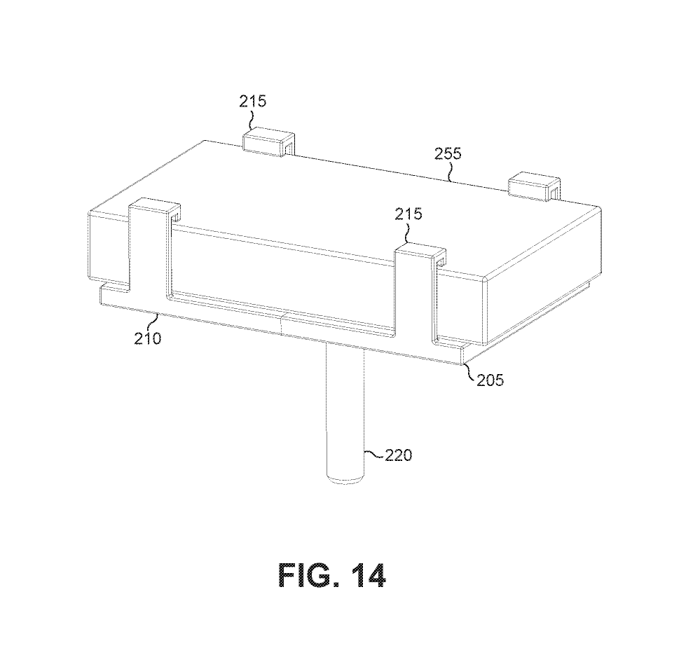

FIGS. 13 and 14 are pictorial diagrams that illustrate insertion of a pharmaceutical injection device into the holder. It should be appreciated that a pharmaceutical injection device, which is also referred to as a wearable injector 255, is brought into the holder 203 with a delivery needle 260 oriented toward the seal-membrane 230. It should be appreciated that, once the penetration needle 225 is removed from the flexible sleeve 220, the seal-membrane 230 substantially reseals itself to reduce the potential for contamination within the volume of the flexible, nonmetallic sleeve 220.

According to one alternative example embodiment of a holder, the holder 203 further includes a retention-clip 215. The retention clip 215, according to this alternative embodiment, snaps in place over the wearable injector 255 in order to restrain such pharmaceutical injection device in a desired position.

FIG. 15 is a pictorial diagram that depicts an underside of one example embodiment of a holder. According to this example embodiment of a holder 203, the holder includes a bottom surface 219. Also included and disposed on the bottom surface 219 is an adhesive surface 285. In those alternative embodiments where in the holder comprises two portions, for example a first portion 210 and a second portion 205, the adhesive surface includes a first adhesive surface 285 disposed on the bottom surface of the first portion 210 of the holder and a second adhesive surface 290 disposed on the bottom surface 219 of the second portion 205 of the holder.

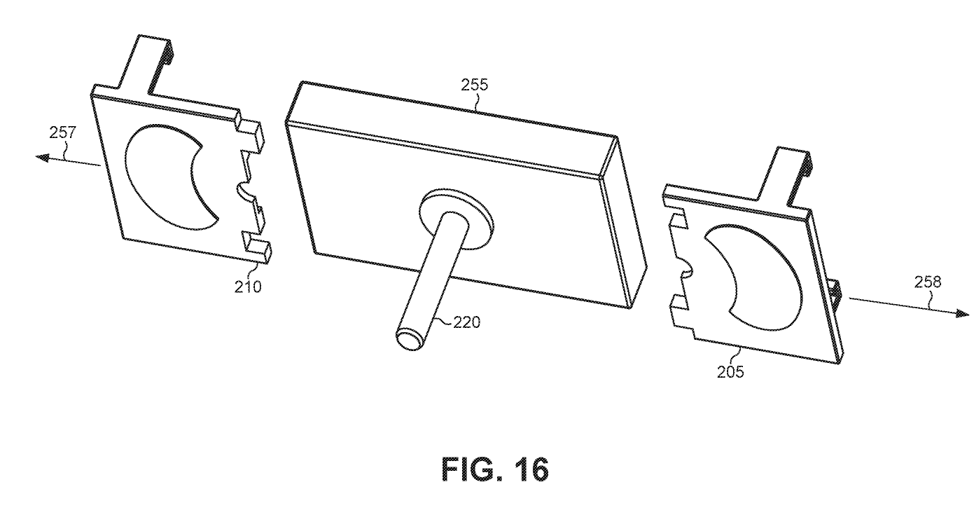

FIG. 16 is a pictorial diagram that illustrates how a flexible sleeve is released from a holder when the holder needs to be replaced. According to yet another alternative example embodiment, the holder also comprises a first portion 210 and a second portion 205 and also comprises the coupling structure as heretofore described. In this alternative example embodiment, the holder 203 provides that the flexible sleeve 220 is loosed from the holder 203 when the first and second portions of the holder are disengaged from each other. It should likewise be appreciated that, according to this alternative example embodiment, as the first portion 210 and second portion 205 of the holder are disengaged from each other, they are pulled apart in substantially opposite directions in order to leave the flexible sleeve 220 engaged with a delivery needle included in a pharmaceutical injection device 255.

While the present method and apparatus has been described in terms of several alternative and exemplary embodiments, it is contemplated that alternatives, modifications, permutations, and equivalents thereof will become apparent to those skilled in the art upon a reading of the specification and study of the drawings. It is therefore intended that the true spirit and scope of the claims appended hereto include all such alternatives, modifications, permutations, and equivalents.

What is claimed is:

1. A method for reducing patient stress during prolonged subcutaneous pharmaceutical injection:
providing a flexible, non-metallic sleeve for a pharmaceutical delivery;
providing a penetration needle within the sleeve;
fixing an upper end of the sleeve in a holder;
penetrating the skin using the sleeve reinforced with the penetration needle;
affixing the holder to a patient's skin;
removing the penetration needle from the sleeve; and
accepting a delivery needle into the sleeve.

2. The method of claim 1 wherein fixing an upper end of the sleeve in a holder comprises:
supporting a lower surface of a collar included in the sleeve with a recessed upper surface of the holder; and
allowing a top surface of the collar to remain substantially flush with an upper surface of the holder.

3. The method of claim 1 wherein providing a penetration needle within the sleeve comprises:
penetrating a barrier disposed over an upper end of the sleeve; and
receiving the penetration needle through the barrier and into the sleeve.

4. The method of claim 1 wherein accepting a delivery needle into the sleeve comprises:
accepting a portion of the delivery needle into the sleeve; and
preventing the received portion of the delivery needle from protruding into a patient.

5. The method of claim 1 wherein accepting a delivery needle into the sleeve comprises:
receiving a portion of the delivery needle substantially contemporaneously with receiving a delivery reservoir into the holder.

6. The method of claim 1 wherein affixing the holder to a patient's skin comprises:
providing an adhesive on a bottom of the holder; and
pressing the adhesive onto the patient's skin.

7. The method of claim 1 further comprising:
substantially sealing between a substantial portion of an outer perimeter of the delivery needle and of an upper end of the sleeve.

8. The method of claim 1 further comprising:
reducing the size of an orifice created in an upper end of the sleeve when the penetration needle is removed.

9. The method of claim 1 further comprising:
receiving a delivery reservoir into the holder; and
restraining the delivery reservoir once it is in the holder.

10. The method of claim 1 further comprising:
maintaining the delivery needle in a substantially similar penetration in the sleeve;
detaching the holder from the sleeve;
attaching a new holder to the sleeve; and
affixing the new holder to the patient's skin.

11. A system for reducing patient stress during prolonged subcutaneous pharmaceutical injection comprising:
holder for receiving a pharmaceutical injection device, said holder including a top surface and a bottom surface and further including an opening for holding a flexible sleeve;
and
adhesive surface disposed on a portion of the bottom surface of the holder.

12. The system of claim 11 further comprising a flexible sleeve disposed in the opening that runs through the top and bottom surfaces and which includes:
upper end, said upper end including a collar for preventing the upper end of the flexible sleeve from moving from the top surface of the holder toward the bottom surface of the holder; and
bottom end for penetration into a patient's skin;

13. The system of claim 12 wherein the flexible sleeve includes a seal-membrane disposed across the upper end, said seal membrane being pliable to allow penetration by a penetration needle.

14. The system of claim 11 wherein the holder comprises a first portion and a second portion and where the interface between the first portion and the second portion includes an interlock structure for substantially maintaining the position of the first portion relative to the second portion when the two portions are coupled together.

15. The system of claim 11 wherein the holder comprises a first portion and a second portion and where the interface between the first portion and the second portion includes a coupling structure for substantially maintaining the position of the first portion relative to the second portion and wherein the flexible sleeve is loosed from the holder when the first and second portions of the holder are disengaged from each other.

16. The system of claim 11 wherein the holder further includes a retention-clip to secure the pharmaceutical injection device into a desired position.

17. The system of claim 16 wherein the holder comprises a first portion and a second portion and where the interface between the first portion and the second portion includes a coupling structure for substantially maintaining the position of the first portion relative to the second portion and wherein the pharmaceutical injection device is loosed from the retention clip when the first and second portions of the holder are disengaged from each other.

18. The system of claim 11 further comprising:
penetration needle that includes:
puncture end for penetrating skin; and
cap end for preventing the puncture needle from protruding by a pre-established amount beyond the bottom end of the sleeve.

19. An adapter to reduce stress of injection comprising:
means for penetrating the skin with a flexible tubing;
means for holding the tubing in a desired position on the patient;
means for securing a delivery reservoir to maintain a portion of a delivery needle coupled to the delivery reservoir in the flexible tubing; and
means for preventing the delivery needle from penetrating a patient beyond a pre-established amount.

20. The adapter of claim 19 wherein the means for holding the tubing includes a means for allowing the flexible tubing to remain engaged with a patient when the holder is removed from the patient.

21. The adapter of claim 19 further comprising a means for penetrating a patient's skin to facilitate penetration of the skin with the flexible tubing.
